(19) United States
(12) Patent Application Publication
(10) Pub. No.: US 2005/0137544 A1
(43) Pub. Date: Jun. 23, 2005

(54) Absorbent article with skin care composition

(75) Inventors: Eric Schroeder, Neenah, WI (US); Lewis P. Orchard IV, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/741,759

(22) Filed: Dec. 18, 2003

(51) Int. Cl. A61F 13/15; A61F 13/20

(52) U.S. Cl. 604/367

(57) ABSTRACT

The present invention relates to absorbent articles having a front half extending from a front edge to a longitudinal centerline, a back half extending from a back edge to a longitudinal centerline and a width defined by a narrowest part of the absorbent article. The absorbent articles may include a skin care composition on at least a portion of the bodyfacing surface of a liner. The skin care composition may have a density of less than or equal to about 3.4×10⁻⁵ g/mm². The absorbent articles may have a front/back ratio of skin care composition of less than or equal to about 0.70.

### BACKGROUND OF THE INVENTION

The present disclosure relates generally to the inclusion of skin care compositions on the bodyfacing materials of disposable absorbent articles.

Disposable absorbent articles such as diapers, training pants, adult incontinence products, absorbent under pants, feminine care products, and nursing pads have been used to absorb body fluids and leave the skin dry. Disposable absorbent articles of this type generally include a liquid impermeable backsheet member, an absorbent core or assembly, and a liquid permeable body facing or liner material. The body facing or liner material comes into contact with the wearer's skin. While the body facing material is made of a soft, compliant material, the material rubs against the skin during use and may not leave the skin completely dry and free of the bodily fluids, such as solid or semi-solid waste that the absorbent article is trying to absorb. During frequent insults of bodily fluids and frequent use of disposable absorbent articles, the skin can become irritated, appear red, and be sore to the touch. Creams, lotions, or ointments can be used to provide an artificial hydrophobic barrier on the skin and to treat skin conditions such as diaper rash. Application of these types of products to the skin is often messy and inconvenient. Often, these products are not used prophylactically and are only used when signs of diaper rash are visible.

Commercially available infant diaper products have been sold that provide a skin care composition on the liner of the diaper in order to provide beneficial effects on the skin of the diaper wearer. For example, a skin care composition (or “lotion” or “ointment”) may be provided on the material contacting the diaper wearer's skin in order to prevent or to reduce the discomfort associated with diaper rash. Specifically, the Procter & Gamble Co. (Cincinnati, Ohio) has sold PAMPERS Rash Care diapers containing hypoallergenic ointment stripes. Packages of such diapers indicate that the ointment contains the following ingredients: white petrolatum, zinc oxide, isononyl isononanoate, polyglycerol-6 polyricinoleate, silica dimethyl silylate, and stearyl alcohol. As another example, the Kimberly-Clark Corporation (Neenah, Wis.) has sold HUGGIES Supreme and Ultratrim diapers containing hypoallergenic ointment stripes.

There are also several U.S. patent references that describe the application of an ointment or lotion onto the body-contacting surface of an absorbent article, such as a diaper. The body-contacting surface may include the “bodyside liner” or “topsheet” of the article. For example, U.S. Pat. No. 3,896,807 to Buchalter describes an article of apparel, such as a diaper, which is to be worn by the user so that an inside portion of the article having a therapeutic cream comes into direct contact with the user's skin. U.S. Pat. No. 5,643,588 to Roe et al. describes a diaper containing a liquid pervious topsheet with a lotion composition. U.S. Pat. No. 6,426,444 to Roe et al. (hereinafter “the '444 patent) also describes a diaper containing a liquid pervious topsheet with a lotion composition. The '444 patent further describes that the lotion composition may be applied to the entire surface of the topsheet or to portions of the topsheet. The '444 patent describes that the lotion composition can be applied in a stripe aligned with and centered on the longitudinal centerline of the article. The '444 patent also describes that some portions of the treated surface of the topsheet can have greater or lesser amounts of lotion composition, including portions of the surface that do not have any lotion composition on them. More specifically, the '444 patent describes a preferred embodiment in which the surface of the topsheet will have regions where no lotion is applied, particularly in areas of the topsheet that correspond to the crotch region of the article. The '444 patent describes the “crotch region” of the article as a rectangle that is centered longitudinally and laterally about the article's crotch point. The '444 patent describes that the length of the crotch region corresponds to 40% of the absorbent article's total length and that the width of the crotch region is equivalent to the width of the widest absorbent core component as measured at the crotch point. Additionally, the '444 patent describes that for certain lotion compositions; the untreated regions further facilitate liquid passage through the topsheet and into the absorbent core. The '444 patent describes that where the lotion is applied nonuniformly, the lotion may have a pattern of discrete dots or stripes that run in the longitudinal or lateral direction of the article.

While the '444 patent describes patterns of lotion application that facilitate liquid passage through the topsheet and into the absorbent core, the '444 patent does not disclose patterns of lotion application that facilitate transfer of lotion to the skin of the article wearer.

### SUMMARY OF THE INVENTION

Disclosed herein are absorbent articles. The absorbent articles of the invention have a length and a width where the length defines a longitudinal direction of the articles and the width defines a lateral direction of the articles. The absorbent articles generally include a front waist region, a back waist region and a crotch region that interconnects the front waist region and the back waist region. The absorbent articles also define two longitudinal edges and two waist edges, a front waist edge and a back waist edge. The absorbent articles of the invention may be considered as having a front half where the front half extends from the front waist edge, or front edge, to a longitudinal centerline. The longitudinal centerline generally divides the length of the absorbent article in half. The absorbent articles of the invention may also have a back half extending from a back waist edge, or back edge, to the longitudinal centerline. The absorbent articles may further include a width defined by a narrowest part of the absorbent article, which may be considered to be the part of the absorbent article where the longitudinal edges come closest to each other, or put differently, have the shortest distance between them in the lateral direction. The width at the narrowest part of the absorbent article is the width at the narrowest part of the article “footprint”, as opposed to the narrowest distance between individual components of the article. The absorbent articles may include the following typical components: an outer cover, a liquid permeable bodyside liner that is connected in superposed relation to the outer cover and an absorbent body that is located between the bodyside liner and the outer cover on a side of the bodyside liner opposite a bodyfacing surface.

The absorbent articles of the invention may further include a skin care composition on at least a portion of the bodyfacing surface. The skin care composition may have a density of less than or equal to about 3.4×10⁻⁵ g/mm². The skin care composition may also have a density of from about 2.4×10⁻⁵ g/mm² to about 3.1×10⁻⁵ g/mm². More specifically, the skin care composition may have a density of from about 2.8×10⁻⁵ g/mm² to about 3.0×10⁻⁵ g/mm². Even more specifically, the skin care composition may have a density of from about 2.8×10⁻⁵ g/mm² to about 2.9×10⁻⁵ g/mm². Alternatively, the skin care composition may have a density of less than or equal to about 2.8×10⁻⁵ g/mm². Additionally, the absorbent articles of the invention may have a front/back ratio of skin care composition of less than or equal to about 0.70. Determination of the “front/back ratio of skin care composition” is described in more detail herein. The front/back ratio of skin care composition may also be less than or equal to about 0.60. Alternatively, the front/back ratio of skin care composition may be less than or equal to about 0.45 or less than or equal to about 0.35.

With other aspects of the present invention, the absorbent articles may have a covered area of the back half, where the percentage of the back half of the article that is covered with skin care composition is greater than about 15%. Alternatively, the covered area may be greater than or equal to about 20%; greater than or equal to about 30%; or greater than or equal to about 40%. With different aspects of the present invention, the skin care composition may include at least two different compositions. Further, the compositions may be located on different portions of the bodyfacing surface of the articles. The absorbent articles of the invention also include articles where the skin care composition has different densities in different areas of the bodyfacing surface.

In another aspect, the present invention includes absorbent articles having a front half that extends from a front edge to a longitudinal centerline of the article. The longitudinal centerline divides the length of the articles in half. The articles also include a back half that extends from a back edge to the longitudinal centerline. The absorbent articles also have a width defined by the narrowest part of the absorbent article. The width at the narrowest part is the portion of the article where the longitudinal sides are closest together (or have the shortest distance between them in the lateral direction), but not necessarily where individual components of the article are closest together. The absorbent articles may include an outer cover, a liquid permeable bodyside liner that is connected in superposed relation to the outer cover and an absorbent body that is located between the bodyside liner and the outer cover. The absorbent body may be located on a side of the bodyside liner opposite a bodyfacing surface. The absorbent articles may also include a skin care composition on at least a portion of the bodyfacing surface. The skin care composition may have a density of less than or equal to about 3.4×10⁻⁵ g/mm². Additionally, the skin care composition may cover greater than or equal to 15% of an area of the back half. Determination of the area of coverage is described in greater detail herein.

These aspects and additional aspects of the invention will be described in greater detail herein. Further, it is to be understood that both the foregoing general description and the following detailed description are exemplary and are intended to provide further explanation of the invention claimed. The accompanying drawings, that are incorporated in and constitute part of this specification, are included to illustrate and provide a further understanding of the disposable absorbent articles of the invention. Together with the description, the drawings serve to explain various aspects of the invention.

### BRIEF DESCRIPTION OF THE DRAWINGS

Referring now to the figures, which are exemplary, not limiting, wherein like elements are numbered alike.

FIG. 1 representatively shows a partially cut away, top plan view of an absorbent article in a stretched and laid flat condition with the surface of the article that contacts the skin of the wearer facing the viewer.

FIG. 2 representatively shows a sectional view of the absorbent article of FIG. 1 taken along line 2-2.

FIG. 3 is a graph showing a plot of percent of skin care composition retained on the liner after converting against the skin care composition density applied in grams per square millimeter (g/mm²) entitled “Skin Care Composition Applied at 155° F.”.

FIG. 4 is a graph showing a plot of the amount of skin care composition transferred from the liner to a substrate in grams against the skin care composition density in grams per square millimeter (g/mm²) entitled “Transfer versus Skin Care Composition Density”.

FIG. 5 representatively shows a top plan view of the bodyside liner of the absorbent article of FIG. 1 with the surface that contacts the wearer facing the viewer and illustrating an embodiment of a skin care composition in a current commercial diaper execution.

FIG. 6 is representatively shows a top plan view of the bodyside liner of the absorbent article of FIG. 1 with the surface that contacts the wearer facing the viewer and illustrating an embodiment of the application design of the skin care composition in a geometric pattern of an “off set” design.

FIG. 7 is representatively shows a top plan view of the bodyside liner of the absorbent article of FIG. 1 with the surface that contacts the wearer facing the viewer and illustrating an embodiment of the application design of the skin care composition in a geometric pattern of a “zoned back” design.

FIG. 8 is representatively shows a top plan view of the bodyside liner of the absorbent article of FIG. 1 with the surface that contacts the wearer facing the viewer and illustrating an embodiment of the application design of the skin care composition in a geometric pattern of a “shaped zoned back” design.

FIG. 9 is representatively shows a top plan view of the bodyside liner of the absorbent article of FIG. 1 with the surface that contacts the wearer facing the viewer and illustrating an embodiment of the application design of the skin care composition in a geometric pattern of a “zoned front/back” design.

FIG. 10 is representatively shows a top plan view of the bodyside liner of the absorbent article of FIG. 1 with the surface that contacts the wearer facing the viewer and illustrating an embodiment of the application design of the skin care composition in a geometric pattern of a “shaped zoned front/back” design.

FIG. 11 is representatively shows a top plan view of the bodyside liner of the absorbent article of FIG. 1 with the surface that contacts the wearer facing the viewer and illustrating an embodiment of the application design of the skin care composition in a geometric pattern of a “shaped zoned front/back matrix” design.

### DETAILED DESCRIPTION OF THE INVENTION

The present disclosure is directed to solving problems related to protecting the barrier function of the skin when the skin is exposed to causes of physical and biological damage, and to the prevention and treatment of diaper rash.

The present disclosure will be expressed in terms of its various components, elements, constructions, configurations, arrangements and other features that may also be individually or collectively be referenced by the term, “aspect(s)”, or other similar terms. It is contemplated that the various embodiments may incorporate one or more of its various features and aspects, and that such features and aspects may be employed in any desired, operative combination.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof. Additionally, all ranges disclosed herein are inclusive and combinable (e.g., the ranges of less than or equal to about 25, with about 5 to about 20 desired, and about the amounts of 10 to about 15 more desired, include the ranges of about 5 to about 25, about 10 to about 25, about 5 to about 15, etc.).

The following detailed description will be made in the context of one type of absorbent article, a disposable diaper that is adapted to be worn by infants about their lower torso. It is readily apparent, however, that the invention would also be suitable for use with other types of absorbent articles, such as incontinence garments (e.g., an adult incontinence garment), training pants, swim pants, feminine hygiene products and prefastened and/or refastenable diaper pants (collectively hereinafter referred to as “diaper”).

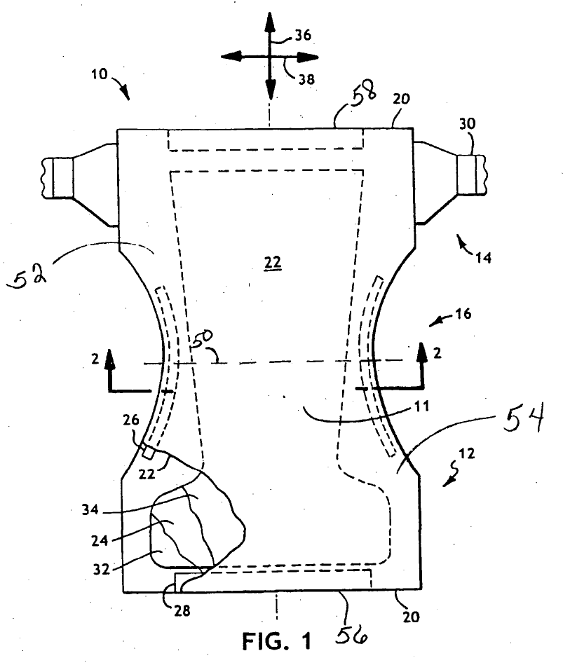

FIG. 1 is a representative plan view of a disposable diaper 10 in a flat-out, uncontracted state (i.e., with all elastic induced gathering and contraction removed). The bodyfacing surface 11 of the diaper 10, that is, the surface 11 of the diaper 10 that contacts the wearer is facing the viewer. Disposed on a portion of the bodyfacing surface 11 is a composition, e.g., a lotion for treating the skin of the wearer. The compositions can be applied to one or more bodyfacing materials that are components of the diaper 10. As used herein, the term “bodyfacing material” includes, but is not limited to, materials such as the bodyside liner or topsheet, and elastic material such as leg elastics, waist elastics and flaps. Each of these materials and components of a diaper 10 are described more fully herein. The compositions are applied to one or more of the bodyfacing materials in order to have a beneficial effect on the skin barrier. The bodyfacing material can be a single layer or multi-layered.

With reference to FIG. 1, the diaper 10 generally defines a front waist section 12, a rear waist section 14, and an intermediate section 16 that interconnects the front and rear waist sections 12 and 14. The front and rear waist sections 12 and 14 include the general portions of the diaper 10 that are constructed to extend substantially over the wearer's front and rear abdominal regions, respectively, during use. The intermediate section 16 of the diaper 10 includes the general portion of the diaper 10 that is constructed to extend through the wearer's crotch region between the legs.

The diaper 10 includes a backsheet or outer cover 20, a liquid permeable topsheet or bodyside liner 22 positioned in facing relation with the outer cover 20, and an absorbent body 24, such as an absorbent pad, which is located between the outer cover 20 and the bodyside liner 22. The outer cover 20 defines a length and a width that, in the illustrated aspect, coincide with the length and width of the diaper 10. The absorbent body 24 generally defines a length and width that are less than the length and width of the outer cover 20, respectively. Thus, marginal portions of the diaper 10, such as marginal sections of the outer cover 20, may extend past the terminal edges of the absorbent body 24. In the illustrated aspects, for example, the outer cover 20 extends outwardly beyond the terminal marginal edges of the absorbent body 24 to form side margins and end margins of the diaper 10. The bodyside liner 22 is generally coextensive with the outer cover 20 but may optionally cover an area that is larger or smaller than the area of the outer cover 20, as desired. In other words, the bodyside liner 22 is connected in superposed relation to the outer cover 20. The outer cover 20 and bodyside liner 22 are intended to face the garment and body of the wearer, respectively, while in use.

To provide improved fit and to help reduce leakage of body exudates from the diaper 10, the diaper side margins and end margins may be elasticized with suitable elastic members, such as single or multiple strands of elastic. The elastic strands may be composed of natural or synthetic rubber and may optionally be heat shrinkable or heat elasticizable. For example, as representatively illustrated in FIG. 1, the diaper 10 may include leg elastics 26 that are constructed to operably gather and shirr the side margins of the diaper 10 to provide elasticized leg bands which can closely fit around the legs of the wearer to reduce leakage and provide improved comfort and appearance. Similarly, waist elastics 28 can be employed to elasticize the end margins of the diaper 10 to provide elasticized waists. The waist elastics 28 are configured to operably gather and shirr the waist sections to provide a resilient comfortably close fit around the waist of the wearer. In the illustrated aspects, the elastic members are illustrated in their uncontracted, stretched condition for the purpose of clarity.

Fasteners 30, such as hook and loop fasteners, may be employed to secure the diaper 10 on a wearer. Alternatively, other fastening means, such as buttons, pins, snaps, adhesive tape fasteners, cohesives, mushroom-and-loop fasteners, and/or the like, may be employed. Additionally, more than two fasteners can be provided, particularly if the diaper 10 is to be provided in a prefastened configuration. The fasteners can vary in size and form.

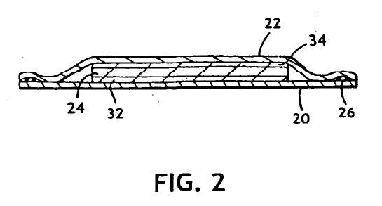

The diaper 10 may further include other layers between the absorbent body 24 and the bodyside liner 22 or outer cover 20. For example, as representatively illustrated in FIGS. 1 and 2, the diaper 10 may include a ventilation layer 32 located between the absorbent body 24 and the outer cover 20 to insulate the outer cover 20 from the absorbent body 24, to improve air circulation and to effectively reduce the dampness of the garment facing surface of the outer cover 20. The ventilation layer 32 may also assist in distributing fluid exudates to portions of the absorbent body 24 that do not directly receive the insult. The diaper 10 may also include a surge management layer 34 located between the bodyside liner 22 and the absorbent body 24 to prevent pooling of the fluid exudates and further improve air exchange and distribution of the fluid exudates within the diaper 10.

The diaper 10 may be of various suitable shapes. For example, the diaper may have an overall rectangular shape, T-shape or an approximately hourglass shape. In the shown aspect, the diaper 10 has a generally I-shape. The diaper 10 further defines a longitudinal direction 36 and a lateral direction 38. Other suitable diaper components that may be incorporated on absorbent articles include containment flaps, waist flaps, elastomeric side panels, and the like. Likewise, if the diaper 10 is to be sold in a prefastened condition, the diaper 10 may have passive bonds (not shown) that join the rear waist section 14 with the front waist section 12.

Examples of diaper configurations suitable for use in connection with the instant application that may include other diaper components suitable for use on diapers are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996 to Hanson et al.

The various components of the diaper 10 are integrally assembled together employing various types of suitable attachment mechanisms, such as adhesive, sonic bonds, thermal bonds, and the like, as well as combinations including at least one of these mechanisms. In the shown aspect, for example, the bodyside liner 22 and outer cover 20 are assembled to each other and to the absorbent body 24 with lines of adhesive, such as a hot melt, pressure-sensitive adhesive. Similarly, other diaper components, such as the elastic members 26 and 28, fastening members 30, and ventilation and surge layers 32 and 34 may be assembled into the diaper 10 by employing the above-identified attachment mechanisms.

The outer cover 20 of the diaper 10, as representatively illustrated in FIG. 1, is composed of a substantially vapor permeable material. The permeability of the outer cover 20 is configured to enhance the breathability of the diaper 10 and to reduce the hydration of the wearer's skin during use without allowing excessive condensation of vapor, such as urine, on the garment facing surface of the outer cover 20 that can undesirably dampen the wearer's clothes. The outer cover 20 is generally constructed to be permeable to at least water vapor and has a water vapor transmission rate of greater than or equal to about 1,000 grams per square meter per 24 hours (g/m²/24 hr), where the water vapor transmission rate test is described in PCT Publication WO 02/051456 published on Jul. 4, 2002. For example, the outer cover 20 may define a water vapor transmission rate of about 1,000 to about 6,000 g/m²/24 hr. Materials that have a water vapor transmission rate less than those above generally do not allow a sufficient amount of air exchange and undesirably result in increased levels of skin hydration.

The outer cover 20 is also desirably substantially liquid impermeable. For example, the outer cover 20 may be constructed to provide a hydrohead value of greater than or equal to about 60 centimeters (cm), desirably greater than or equal to about 80 cm, and more desirably greater than or equal to about 100 cm when subjected to the Hydrostatic Pressure Test described in PCT Publication WO 02/051456 published on Jul. 4, 2002. Materials that have hydrohead values less than those above undesirably result in the strike through of liquids, such as urine, during use. Such fluid strike through can undesirably result in a damp, clammy feeling on the outer cover 20 during use.

The outer cover 20 may include any suitable materials that either directly provide the above desired levels of liquid impermeability and air permeability or, in the alternative, materials that can be modified or treated in some manner to provide such levels. In one aspect, the outer cover 20 may be a nonwoven fibrous web constructed to provide the required level of liquid impermeability. For example, a nonwoven web composed of spunbond or meltblown polymer fibers may be selectively treated with a water repellent coating or laminated with a liquid impermeable, vapor permeable polymer film to provide the outer cover 20. In a particular aspect, the outer cover 20 may include a nonwoven web composed of a plurality of randomly deposited hydrophobic thermoplastic meltblown fibers that are sufficiently bonded or otherwise connected to one another to provide a substantially vapor permeable and substantially liquid impermeable web. The outer cover 20 may also include a vapor permeable nonwoven layer that has been partially coated or otherwise configured to provide liquid impermeability in selected areas.

Examples of suitable materials for the outer cover 20 are also described in U.S. Pat. No. 5,482,765 issued Jan. 9, 1996 in the name of Bradley et al. and entitled “NONWOVEN FABRIC LAMINATE WITH ENHANCED BARRIER PROPERTIES”; U.S. Pat. No. 5,879,341 issued Mar. 9, 1999 in the name of Odorzynski et al. and entitled “ABSORBENT ARTICLE HAVING A BREATHABILITY GRADIENT”; U.S. Pat. No. 5,843,056 issued Dec. 1, 1998, in the name of Good et al. and entitled “ABSORBENT ARTICLE HAVING A COMPOSITE BREATHABLE BACKSHEET”; and U.S. Pat. No. 6,075,179 issued Jun. 13, 2000, in the name of McCormack et al. and entitled “LOW GAUGE FILMS AND FILM/NONWOVEN LAMINATES”.

In another embodiment, the outer cover 20 is provided by an extensible material. Further, the outer cover 20 can also be provided by a material having stretch in both the longitudinal 36 and lateral 38 directions. When the outer cover 20 is made from extensible or stretchable materials, the diaper 10 provides additional benefits to the wearer including improved fit. Examples of absorbent articles including extensible outer covers are described in U.S. Pat. No. 6,503,236 issued Jan. 7, 2003, in the name of Uitenbroek et al. and entitled “ABSORBENT ARTICLE HAVING AN EXTENSIBLE OUTER COVER WITH ACTIVATABLE ZONED BREATHABILITY”, the disclosure of which is herein incorporated by reference.

The bodyside liner 22, as representatively illustrated in FIGS. 1 and 2, defines a bodyfacing surface 11 that is compliant, soft feeling, and nonirritating to the wearer's skin. Further, the bodyside liner 22 may be less hydrophilic than the absorbent body 24, to present a relatively dry surface to the wearer, and may be sufficiently porous to be liquid permeable, permitting liquid to readily penetrate through its thickness. A suitable bodyside liner 22 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 22 is suitably employed to help isolate the wearer's skin from liquids held in the absorbent body 24.

Various woven and nonwoven fabrics can be used for the bodyside liner 22. For example, the bodyside liner 22 may be composed of a meltblown or spunbond web of polyolefin fibers. The bodyside liner 22 may also be a bonded-carded web composed of natural and/or synthetic fibers. The bodyside liner 22 may include a substantially hydrophobic material, and the hydrophobic material may, optionally, be treated with a surfactant or otherwise processed to impart a desired level of wettability and hydrophilicity. In a particular aspect, the bodyside liner 22 includes a nonwoven, spunbond, polypropylene fabric composed of about 2.8 to about 3.2 denier fibers formed into a web having a basis weight of about 22 grams per square meter (g/m²) and a density of about 0.06 gram per cubic centimeter (g/cc).

In a particular aspect, the bodyside liner 22 may be surface treated with about 0.3 weight percent (wt %) of a surfactant mixture that contains a mixture of AHCOVEL Base N-62 surfactant and GLUCOPON 220UP surfactant in about a 3:1 ratio based on a total weight of the surfactant mixture. The AHCOVEL Base N-62 surfactant is purchased from Hodgson Textile Chemicals Inc., a business having offices in Mount Holly, N.C., and includes a blend of hydrogenated ethoxylated castor oil and sorbitan monooleate in a 55:45 weight ratio. The GLUCOPON 220UP surfactant is purchased from Henkel Corporation and includes alkyl polyglycoside. The surfactant may also include additional ingredients such as aloe. The surfactant may be applied by various methods, such as spraying, printing, brush coating, foam or the like. The surfactant may be applied to the entire bodyside liner 22 or may be selectively applied to particular sections of the bodyside liner 22, such as the medial section along the longitudinal centerline of the diaper, to provide greater wettability of such sections.

The absorbent body 24 of the diaper 10, as representatively illustrated in FIG. 1, may suitably comprise a matrix of hydrophilic fibers, such as a web of cellulosic fluff, mixed with particles of a high-absorbency material commonly known as superabsorbent material. In a particular aspect, the absorbent body 24 includes a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent body 24 may include a laminate of fibrous webs and superabsorbent material or other suitable matrix for maintaining a superabsorbent material in a localized area.

The absorbent body 24 may have any of a number of shapes. For example, the absorbent body 24 may be rectangular, 1-shaped, or T-shaped. It is generally preferred that the absorbent body 24 is narrower in the intermediate section than in the front or rear waist sections of the diaper 10. The absorbent body 24 may be provided by a single layer or, in the alternative, may be provided by multiple layers, all of which need not extend the entire length and width of the absorbent body 24. In a particular aspect, the absorbent body 24 can be generally T-shaped with the laterally extending cross-bar of the “T” generally corresponding to the front waist section 12 of the absorbent article for improved performance, especially for male infants. In the illustrated aspects, for example, the absorbent body 24 across the front waist section 12 of the article has a cross-directional width of about 18 centimeters (cm), the narrowest portion of the intermediate section 16 has a width of about 7.5 centimeters, and in the rear waist section 14 has a width of about 11.4 centimeters.

The size and the absorbent capacity of absorbent body 24 should be compatible with the size of the intended wearer and the liquid loading imparted by the intended use of the absorbent article. Further, the size and the absorbent capacity of the absorbent body 24 can be varied to accommodate wearers ranging from infants through adults. In addition, it has been found that the densities and/or basis weights of the absorbent body 24 can be varied. In a particular aspect, the absorbent body 24 has an absorbent capacity of greater than or equal to about 300 grams of synthetic urine.

In aspects wherein the absorbent body 24 includes the combination of hydrophilic fibers and high-absorbency particles, the hydrophilic fibers and high-absorbency particles can form an average basis weight for the absorbent body 24 that is about 400 to about 900 g/m². In certain aspects, the average composite basis weight of such an absorbent body 24 is about 500 to about 800 g/m², and preferably is about 550 to about 750 g/m² to provide the desired performance.

To provide the desired thinness dimension to the various configurations of the absorbent article, the absorbent body 24 can be configured with a bulk thickness that is less than or equal to about 0.6 centimeters. Preferably, the bulk thickness is not more than about 0.53 centimeters, and more preferably is less than or equal to about 0.5 centimeters to provide improved benefits. The bulk thickness is determined under a restraining pressure of 0.2 pounds per square inch (psi; 1.38 kiloPascals (kPa)).

The high-absorbency material can be selected from natural, synthetic, and modified natural polymers and materials. The high-absorbency materials can be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent body 24 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. In general, the high absorbency material is present in the absorbent body 24 in an amount of about 5 to about 90 wt %, desirably in an amount of greater than or equal to about 30 wt %, and even more desirably in an amount of greater than or equal to about 50 wt % based on a total weight of the absorbent body 24. For example, in a particular aspect, the absorbent body 24 may include a laminate that includes greater than or equal to about 50 wt % and desirably greater than or equal to about 70 wt % of high-absorbency material overwrapped by a fibrous web or other suitable material for maintaining the high-absorbency material in a localized area.

An example of high-absorbency material is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

Optionally, a substantially hydrophilic tissue or nonwoven wrapsheet (not illustrated) may be employed to help maintain the integrity of the structure of the absorbent body 24. The tissue wrapsheet is typically placed about the absorbent body 24 over at least the two major facing surfaces thereof. The tissue wrapsheet can be composed of an absorbent cellulosic material, such as creped wadding or a high wet-strength tissue. In one aspect, the tissue wrapsheet can be configured to provide a wicking layer that helps to rapidly distribute liquid over the mass of absorbent fibers constituting the absorbent body 24.

Due to the thinness of absorbent body 24 and the high absorbency material within the absorbent body 24, the liquid uptake rates of the absorbent body 24, by itself, may be too low, or may not be adequately sustained over multiple insults of liquid into the absorbent body 24. To improve the overall liquid uptake and air exchange, the diaper 10 of the different aspects may further include a porous, liquid-permeable layer of surge management material 34, as representatively illustrated in FIG. 1. The surge management layer 34 is typically less hydrophilic than the absorbent body 24, and has an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the diaper 10 positioned against the wearer's skin, thereby reducing the feeling of wetness by the wearer. The structure of the surge management layer 34 also generally enhances the air exchange within the diaper 10.

Various woven and nonwoven fabrics can be used to construct the surge management layer 34. For example, the surge management layer 34 may be a layer composed of a meltblown or spunbond web of synthetic fibers, such as polyolefin fibers. The surge management layer 34 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge management layer 34 may be composed of a substantially hydrophobic material, and the hydrophobic material may optionally be treated with a surfactant or otherwise processed to impart a desired level of wettability and hydrophilicity. In a particular aspect, the surge management layer 34 includes a hydrophobic, nonwoven material having a basis weight of about 30 to about 120 g/m².

Additional materials suitable for the surge management layer 34 are set forth in U.S. Pat. No. 5,486,166 issued Jan. 23, 1996 in the name of Bishop et al. and entitled “FIBROUS NONWOVEN WEB SURGE LAYER FOR PERSONAL CARE ABSORBENT ARTICLES AND THE LIKE”; U.S. Pat. No. 5,490,846 issued Feb. 13, 1996 in the name of Ellis et al. and entitled “SURGE MANAGEMENT FIBROUS NONWOVEN WEB FOR PERSONAL CARE ABSORBENT ARTICLES AND THE LIKE”; and U.S. Pat. No. 5,364,382 issued Nov. 15, 1994 in the name of Latimer et al. and entitled “ABSORBENT STRUCTURE HAVING IMPROVED FLUID SURGE MANAGEMENT AND PRODUCT INCORPORATING SAME”.

As representatively illustrated in FIG. 1, the diaper 10 may also include a ventilation layer 32 located between the outer cover 20 and the absorbent body 24. The ventilation layer 32 serves to facilitate the movement of air within and through the diaper 10 and to prevent the outer cover 20 from being in surface to surface contact with at least a portion of the absorbent body 24. Specifically, the ventilation layer 32 serves as a conduit through which air and water vapor can move from the absorbent body 24 through the vapor permeable outer cover 20.

The ventilation layer 32 may be formed from materials described above as being suitable for the surge management layer 34 such as nonwoven, (e.g., spunbond, meltblown or carded), woven, or knitted fibrous webs composed of natural fibers and/or synthetic polymeric fibers. Suitable fibers include, for example, acrylic fibers, polyolefin fibers, polyester fibers, and the like, as well as blends including at least one of the foregoing fibers. The ventilation layer 32 may also be formed from a porous foam material such as an open-celled polyolefin foam, a reticulated polyurethane foam, and the like. The ventilation layer 32 may include a single layer of material or a composite of two or more layers of material. In a particular aspect, the ventilation layer 32 includes a hydrophobic, nonwoven material having a thickness of greater than or equal to about 0.10 centimeters determined under a restraining pressure of 0.05 psi (0.34 kPa) and a basis weight of about 20 to about 120 g/m². For example, the ventilation layer 32 may include a bonded-carded-web, nonwoven fabric that includes bicomponent fibers and that defines an overall basis weight of about 83 g/m². The ventilation layer 32 in such a configuration can be a homogeneous blend composed of about 60 wt % polyethylene/polyester (PE/PET), sheath-core bicomponent fibers that have a fiber denier of about 3 d and about 40 wt % single component polyester fibers that have a fiber denier of about 6 d and that have fiber lengths of about 3.8 to about 5.08 cm.

The ventilation layer 32 can have any desired shape. Suitable shapes include for example, circular, rectangular, triangular, trapezoidal, oblong, dog-boned, hourglass-shaped, oval, and the like. The ventilation layer 32 may extend beyond, completely over or partially over the absorbent body 24. For example, the ventilation layer 32 may suitably be located over the intermediate section 16 of the diaper 10 and be substantially centered side-to-side. It is generally desired that the entire absorbent body 24 be overlaid with the ventilation layer 32 to prevent substantially all surface to surface contact between the outer cover 20 and the absorbent body 24. In the illustrated aspects, the ventilation layer 32 is coextensive with the absorbent body 24. This allows for the maximum degree of air exchange with minimal dampness on the garment facing surface of the outer cover 20.

In the illustrated aspects, the ventilation layer 32 is arranged in a direct, contacting liquid communication with the absorbent body 24. The ventilation layer 32 may be operably connected to the outer cover 20 with a pattern of adhesive, such as a swirl adhesive pattern. In addition, the ventilation layer 32 may be operably connected to the absorbent body 24 with a pattern of adhesive. The amount of adhesive add-on should be sufficient to provide the desired levels of bonding, but should be low enough to avoid excessively restricting the movement of air and vapor from the absorbent body 24 and through the outer cover 20.

The preceding detailed description relates to the components of the diaper 10. In order to provide the desired barrier and protective properties, the diapers 10 may also include skin care compositions. Preferred skin care compositions are those having the ability to remain on the surface of the top sheet and their resistance to migration into the diaper 10 such that they can readily be transferred to the wearer's skin. It is particularly desirable for the skin care composition to resist migration through the top sheet toward the absorbent body 24.

A skin care composition may be applied to the bodyfacing surface 11 of the bodyside liner 22 of the diaper 10. The compositions can be in a variety of physical forms including emulsions, lotions, creams, ointments, salves, suspensions, encapsulations, gels, hybrids of these forms, and the like, as well as combinations including at least one of these forms. The various embodiments of disposing the composition on the bodyfacing layer 11 are not limited to the type of composition. Exemplary compositions may include hydrophilic solvent(s) or non-aqueous systems alone or in combination with other material(s) such as: high molecular weight polyethylene glycol(s) (i.e., that provide the hydrophilic solvent(s) and any active ingredients in solid form at room temperature), fatty alcohol(s), fatty acid(s), surfactant(s), fat(s), oil(s), sterol(s), and sterol derivative(s), emollient(s), viscosity enhancer(s), rheology modifier(s), solidifying agent(s) (e.g., that render the composition solid at room temperature, and have a penetration hardness of greater than or equal to about 5 millimeters (mm) and a melting point of at least about 32° C.), extracted botanical additive(s) (containing chemically active components of various plants and plant substances), a diaper rash skin protectant (e.g., drug products that protect injured or exposed skin or mucous membrane surface from harmful or annoying stimuli), and additional ingredients, as well as combinations including at least one hydrophilic solvent(s) or non-aqueous system(s) and at least one other material.

Possible additional ingredients include: antifoaming agents (reduce the tendency of foaming during processing); antimicrobial actives; antifungal actives; antiseptic actives; antioxidants (product integrity); antioxidants-cosmetic (reduce oxidation); astringents-cosmetic (induce a tightening or tingling sensation on skin); astringent-drug (a drug product that checks oozing, discharge, or bleeding when applied to skin or mucous membrane and works by coagulating protein); biological additives (enhance the performance or consumer appeal of the product); colorants (impart color to the product); deodorants (reduce or eliminate unpleasant odor and protect against the formation of malodor on body surfaces); other emollients (help to maintain the soft, smooth, and pliable appearance of the skin by their ability to remain on the skin surface or in the stratum corneum to act as lubricants, to reduce flaking, and to improve the skin's appearance); external analgesics (a topically applied drug that has a topical analgesic, anesthetic, or antipruritic effect by depressing cutaneous sensory receptors, or that has a topical counterirritant effect by stimulating cutaneous sensory receptors); film formers (to hold active ingredients on the skin by producing a continuous film on skin upon drying); fragrances (consumer appeal); silicones/organomodified silicones (protection, water resistance, lubricity, softness); oils (mineral, vegetable, and animal); Natural Moisturizing Factor (NMF) and other skin moisturizing ingredients; opacifiers (reduce the clarity or transparent appearance of the product); powders (enhance lubricity, oil adsorption, provide skin protection, astringency, opacity, etc.); skin conditioning agents; solvents (liquids employed to dissolve components found useful in the cosmetics or drugs); and surfactants (such as cleansing agents, emulsifying agents, solubilizing agents, and suspending agents), as well as combinations including at least one of the foregoing additional ingredients.

Moreover, to provide the improved stability and transfer to the skin of the wearer, the compositions may define a melting point of about 32° C. to about 100° C., desirably about 35° C. to about 80° C., and more desirably about 40° C. to about 75° C. The melting points of the compositions cause the compositions to be relatively immobile and localized on the bodyfacing surface 11 of the diaper 10 at room temperature and readily transferable to the skin of the wearer at body temperatures. Desirably, the compositions are easily transferable to the skin by way of normal contact, wearer motion, adhesion or body heat or any combination thereof.

The composition may have a low shear viscosity of about 50,000 to about 1,000,000 centipoise, desirably about 100,000 to about 800,000 centipoise, and more desirably about 300,000 to about 500,000 centipoise for reduced migration and improved transfer to the skin of the wearer. Further, to provide the improved stability and transfer to the skin of the wearer, the compositions may also define a high shear viscosity of less than or equal to about 5,000 centipoise, desirably about 100 to about 500 centipoise, and more desirably about 150 to about 250 centipoise at a temperature of about 60° C. (or higher temperatures depending on the components and melting point of the composition).

The penetration hardness of the compositions may be about 5 to about 365 millimeters (mm), more desirably about 10 to about 300 mm, more desirably about 20 to about 200 mm, and still more desirably about 40 to about 120 mm as measured using ASTM method D 1321.

One or more skin care composition(s) are applied to particular sections of the bodyfacing surface 11, such as the medial section along the longitudinal centerline of the diaper 10, to provide greater lubricity of such sections and to transfer such composition(s) to the wearer's skin. In order to prevent the composition from adversely affecting the ability of the bodyside liner 22 to absorb an insult, the composition is preferably disposed (amount and geometry of disposition), with limited intrusion into the target insult area (e.g., the area generally defined by the surge management layer 34). The amount of composition disposed on the bodyfacing surface 11, as well as the surface area of the disposition, is based upon a sufficient quantity of composition and adequate composition transfer to the wearer's skin. Desirably, the composition is applied to greater than or equal to about 5 percent (%) and more desirably greater than or equal to about 25 percent of the area of the bodyfacing surface 11 of the bodyside liner 22. The composition can be applied to the bodyside liner 22 at any level that provides the desired transfer benefit without substantially inhibiting transfer of fluid(s) through the bodyside liner 22. For example, the total loading of the composition can be about 10 milligrams (mg) to about 1,000 mg per diaper, desirably about 50 mg to about 400 mg, and more desirably about 80 mg to about 200 mg. The loading will depend upon the desired effect of the composition on the skin barrier function, the specific composition, and product size.

The composition may be applied to the bodyside liner 22 in a variety of manners. Methods to uniformly apply the composition to the bodyfacing surface 11 of the bodyside liner 22 include spraying, slot coating, roto-gravure or flexographic printing, foam application, continuous and intermittent roll application, as well as various combinations of application methods. The compositions can be applied after the bodyfacing material has been incorporated into the absorbent article or prior to incorporating the body facing material into the absorbent article.

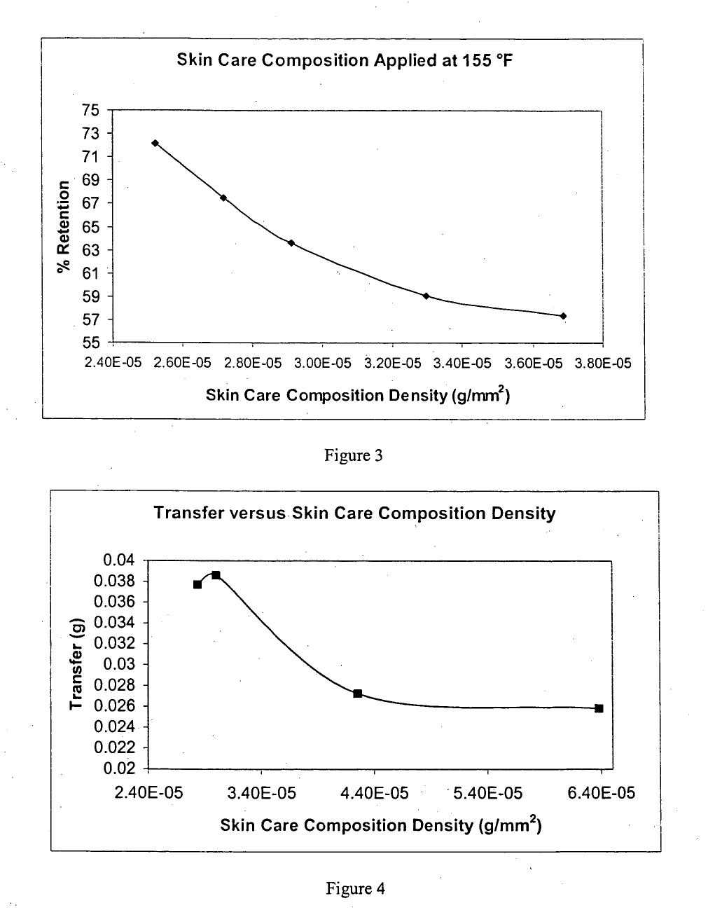

FIG. 3 is a graph showing the relationship between percent of the skin care composition retained by the liner versus the skin care composition density. The graph was built by varying the amount of skin care composition that was applied to the bodyfacing surface of a liner, while keeping the skin care composition area constant. The graph shows that as the skin care composition density is decreased, more skin care composition is retained on the bodyside liner 22. In other words, during the manufacture of the diaper, a greater amount of skin care composition is retained on the liner at skin care composition densities of less than or equal to about 2.7×10⁻⁵ g/mm² than at greater skin care composition densities. As is illustrated, less than 60% of the skin care composition is retained on the diaper bodyside liner (i.e., not transferred to the manufacturing equipment or passed into the absorbent core) at densities of greater than 3.2×10⁻⁵ g/mm², while at densities of less than or equal to about 2.8×10⁻⁵ g/mm², a retention of greater then 65% is obtained, with retentions exceeding 70% at densities of about 2.6×10⁻⁵ g/mm² to 2.8×10⁻⁵ g/mm².

An ink rub procedure was performed to simulate the skin care composition transfer to the wearer's skin. The Ink Rub Tester (Model #10-18-01, available from Testing Machines Inc. (TMI), having offices in Islandia, N.Y.) is an instrument used as a fast and reliable means of assessing the transfer amount of skin care composition from liner to baby's skin by gravimeteric means. Generally, the instrument functions by rubbing a block, covered with a receptor material, against a stable base covered in the treated liner material. The act of rubbing is intended to simulate the movement of the baby's skin against the bodyfacing surface of the diaper liner.

For generating the data in FIG. 3, the Ink Rub Tester (IRT) was used as prescribed in the instruction manual which accompanied the IRT from TMI, with the following exceptions: the receptor material was Natural Silk Noil (Style #651) made by Testfabrics, Inc., and was cut to a sample size of 4 inches by 6 inches (10.2 centimeters (cm) by 15.2 cm). The base material used in the Examples was a spun bond liner treated with centered skin care composition stripes of varying widths and spacing. The base material was dimensioned to approximately 6 inches by 9 inches (15.2 cm by 22.9 cm), merely so that it covered the 6 inch by 9 inch (15.2 cm by 22.9 cm) rubber pad. The slot-coated skin care composition lines were aligned lengthwise and centered as much as possible. The approximately four pound weighted block of the IRT was selected for use during experimentation.

Prior to placing the receptor material on the IRT, the receptor silk material was weighed and its initial mass recorded. The material was folded in a tri-fold so as to ensure that all the material remained on top of the weighing plate. Care was also taken to ensure that no hanging threads existed, as those were likely to be torn off during operation of the instrument and influence later calculations. Additionally, examination gloves were worn at all times to prevent transfer of skin oils to the material, thereby avoiding the creation of a faulty calculation of the change in mass.

Each material was then secured to the IRT. The receptor material was spread out over the weighted block and was held to the block by four magnets, with two magnets evenly spaced on either end of the block. Uniform tension throughout the receptor material was sought, with the material being held tightly with no gaps between the block and the fabric. However, extraordinary strain was not placed on the material. The base material, or liner, was centered upon the rubber pad. Attempts were made to remove all wrinkles in the liner, by spreading the fabric and attaching clips at either end in order to prevent further movement of the fabric. The IRT was set to cycle 50 times at a rate of 100 cycles per minute. Once the prepared materials were secured to the IRT, the device was started and allowed to cycle. At the completion of the selected number of cycles, the liner was removed and thrown away, while the receptor material was removed and was again tri-folded and its weight recorded. The amount of transfer is defined as the difference between the initial and final weight of the receptor material. Generally, the procedure was repeated 10 times to obtain a statistically significant average amount of material transferred.

FIG. 4 is a graph showing the unexpected relationship between the skin care composition density and the amount of skin care composition transferred to the wearer's skin. As can be seen from the graph, the amount of composition transferred has an inverse relationship to the density of the composition. The graph illustrates that, as the skin care composition density decreases, the amount of skin care composition transferred to the wearer's skin increases. In this case, the surface area covered by the skin care composition (e.g., ointment) was increased at constant skin care composition addon. Therefore, the density was decreased by increasing the area covered by skin care composition. It was discovered that, as the density increased, the actual amount of the skin care composition transferred to the skin decreased. The transfer of the composition was less effective at higher densities. The maximum transfer was attained at a density of about 2.9×10⁻⁵ g/mm² to about 3.4×10⁻⁵ g/mm². As can be seen from the graph, a minimum transfer appears to be reached at a density of about 4.4×10⁻⁵ g/mm². Based upon this information, and the retention information provided in FIG. 2, the density of the skin care composition on the diaper is desirably less than or equal to about 3.4×10⁻⁵ g/mm², with less than or equal to about 2.8×10⁻⁵ g/mm² more desired, while about 2.8×10⁻⁵ g/mm² to about 3.0×10⁻⁵ g/mm² is even more desired for some applications.

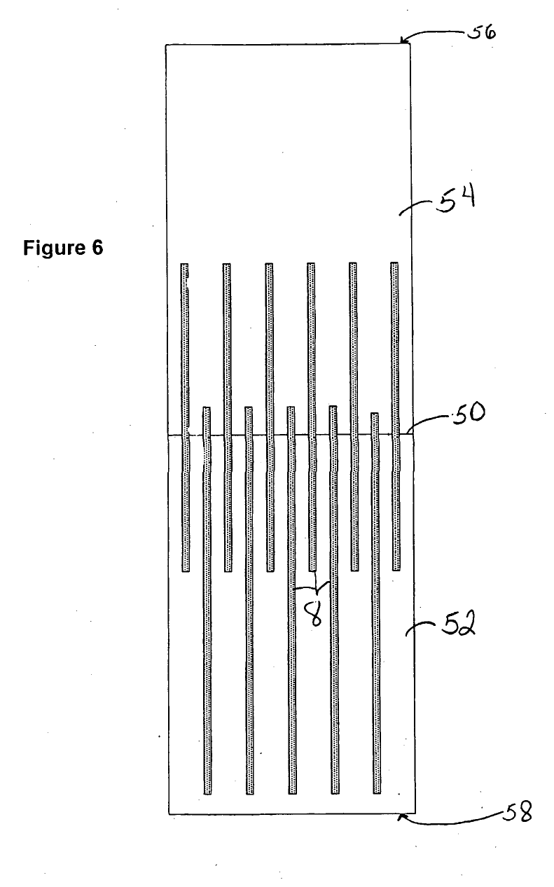

As described earlier, the bodyside liner may or may not extend the full width of the absorbent article. For illustration purposes, the bodyside liner shown in FIGS. 6-11 is meant to extend the full width of the narrowest portion of the diaper (generally in the crotch region) when in a laid flat, non elasticized configuration. As described previously herein, the narrowest portion is the portion where the longitudinal sides of the diaper come closest to each other (or have the shortest distance between them in the lateral direction). In calculating the area percentage coverage of the skin care composition in the examples below, it is this width, the narrowest part of the projected image of the diaper, which is used. Thus, the total area of the front half and back half are equal, and rectangular.

To calculate the area percentage covered of skin care composition in the back half of the absorbent article (e.g., diaper), the length of the back half of the diaper is measured from the back edge 58 to the longitudinal centerline 50. The width is measured from the narrowest portion of the diaper when in a laid flat, nonelasticized configuration, whether this is in the front or back half of the diaper. This length and width defines the area of the back half of the diaper. The area covered by the skin care composition in the back half of the diaper is determined. The area percentage covered of skin care composition in the back half of the diaper is the area covered by the skin care composition in the back half of the diaper multiplied by 100 and divided by the area of the back half of the diaper. A similar determination is made for the area percentage covered of skin care composition in the front half of the absorbent article (e.g., diaper) using the front edge 56 to the longitudinal centerline 50 as the length and the same width as for the back. To calculate the front/back ratio of skin care composition, divide the area percentage covered of skin care composition in the front half of the diaper by the area percentage covered of skin care composition in the back half of the diaper.

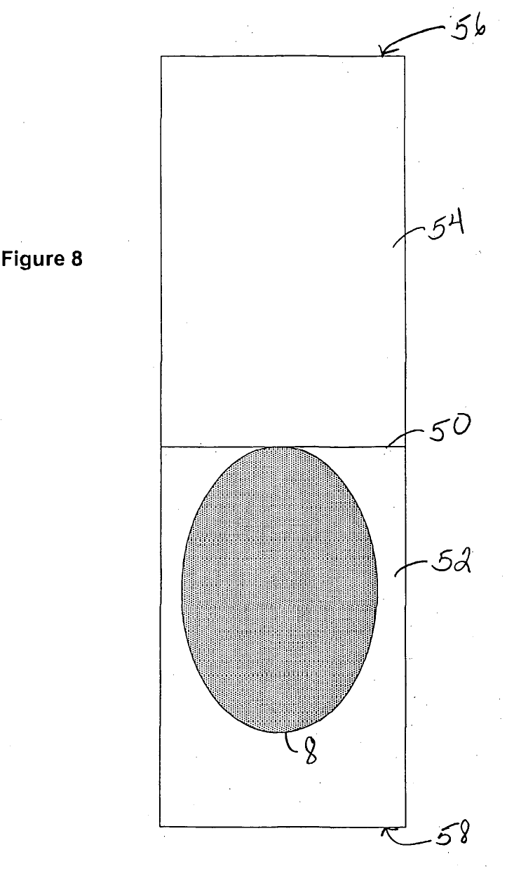

The patterns illustrated in FIGS. 6-11 are designed to enhance transfer (i.e., the amount of composition transferred to the wearer as well as the location of the transferred composition on the wearer's skin), efficiency of transfer, and aesthetics of the absorbent articles. As will be evident from the following figures, various embodiments of the pattern of distribution of the composition are contemplated. The patterns of distribution can be any rounded (e.g., round, elliptical, oval, kidney, a FIG. 8, and the like, including irregular shapes) or polygonal (e.g., rectangular, square, trapezoidal, triangular, hexagonal, octagonal, and the like, including irregular shapes) geometry or a combination including at least one of these geometries wherein the desired amount of composition is disposed within and outside of the intake area. With respect to aesthetics, various designs can be formed, particularly in the back half of the diaper that render the diaper aesthetically pleasing to the wearer and/or the purchaser, while attaining the desired transfer and intake. In all of these figures, the composition is illustrated as element 8 for simplicity.

In one embodiment, the skin care composition is applied on the bodyside liner 22 in a geometric pattern of an “offset” design as shown in FIG. 6. In the “offset” design, the skin care composition is applied in the form of longitudinal stripes beginning in the back half 52 of the absorbent article and ending in the front half 54 of the absorbent article. As can be seen from this design, the majority of the skin care composition is disposed in the back half of the diaper such that the skin care composition will contact and transfer to the anal region of the wearer. In one aspect of the present pattern of distribution, the area coverage in the back of the diaper is 19%. The front to back area ratio is 0.42.

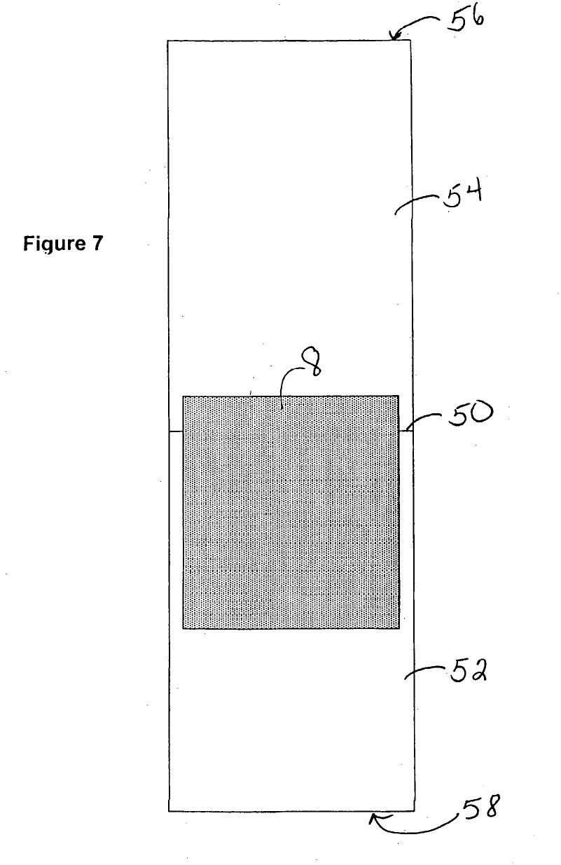

In yet other aspects, the skin care composition is applied on the bodyside liner 22 of the absorbent article in a geometric pattern of a “zoned back” design as shown in FIG. 7 and a “shaped zoned back” design as shown in FIG. 8. In the “zoned back” design, the skin care composition is applied in the form of a rectangle primarily in the back half 52 of the absorbent article, while in the “shaped zoned back” design, the skin care composition is applied in an oval shape in the back half 52 of the absorbent article. It is noted that these are merely exemplary and any number of shapes and geometric patterns may be employed. Both of these designs minimize interference with intake and maximize the contact area between the skin and the skin care composition in the anal area. In the “zoned back” aspect of the present pattern of distribution, the area coverage in the back of the diaper is 46%. The front to back area ratio is 0.15. In the “shaped zoned back” aspect of the present pattern of distribution, the area coverage in the back of the diaper is 47%. The front to back area ratio is 0.

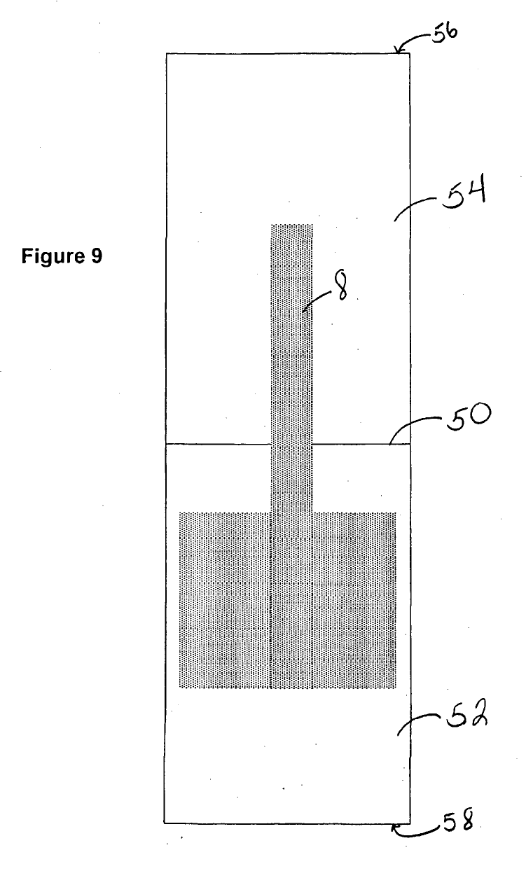

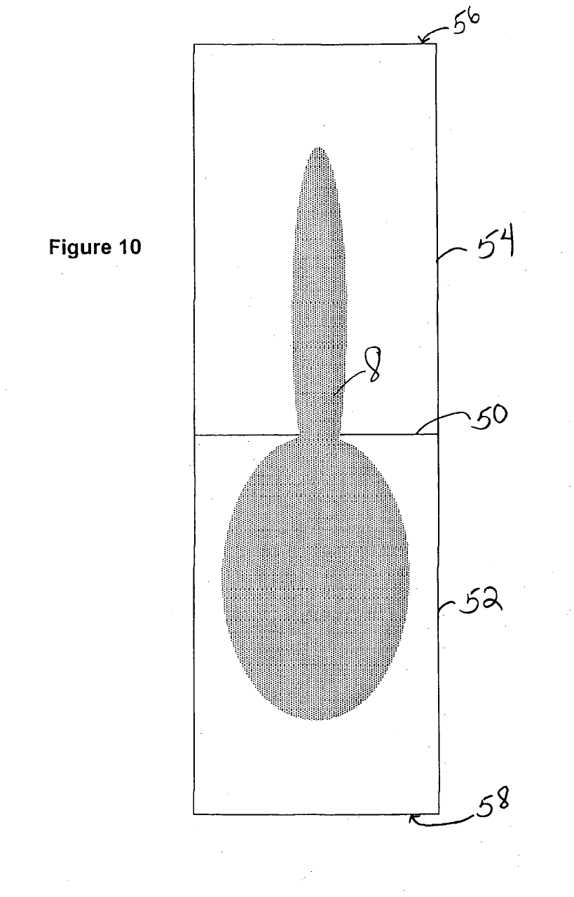

With the aspects illustrated in FIGS. 9 and 10, the skin care composition is applied on the bodyside liner 22 of the absorbent article in geometric patterns of a “zoned front/back” and “shaped zoned front/back”, respectively. In the “zoned front/back” design, the skin care composition may be applied in the form of a square in the back half 52 of the absorbent article with a longitudinal stripe of the skin care composition extending from the rectangle into the front half 54 of the absorbent article. In the “shaped zoned front/back” design, the skin care composition is applied in an oval shape in the back half 52 of the absorbent article with a longitudinal stripe of the skin care composition extending into the front half 54 of the absorbent article. These designs allow for more ubiquitous coverage while minimizing interference with intake in the front of the diaper. In the “zoned front/back” embodiment of the present pattern of distribution, the area coverage in the back of the diaper is 43%. The front to back area ratio is 0.23. In the “shaped zoned front/back” embodiment of the present pattern of distribution, the area coverage in the back of the diaper is 46%. The front to back area ratio is 0.30.

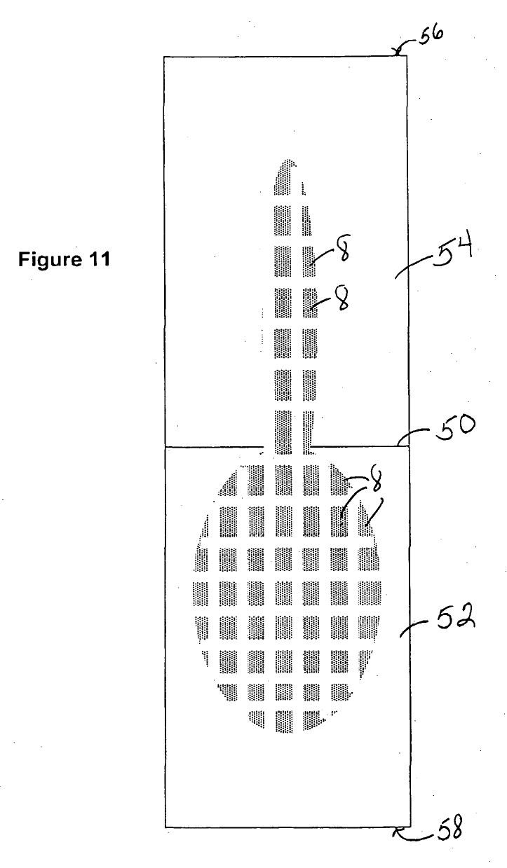

In the embodiment illustrated in FIG. 11, the skin care composition is applied on the bodyside liner 22 of the absorbent article in a matrix of intersecting lines of skin care composition generally following the pattern of the “shaped zoned front/back” design of FIG. 10. In this embodiment, “shaped zoned/front/back matrix” the coverage area is reduced by the alternating regions with no skin care composition within the matrix. In the “shaped zoned front/back matrix” embodiment of the present pattern of distribution, the area coverage in the back of the diaper is 23%. The front to back area ratio is 0.30.

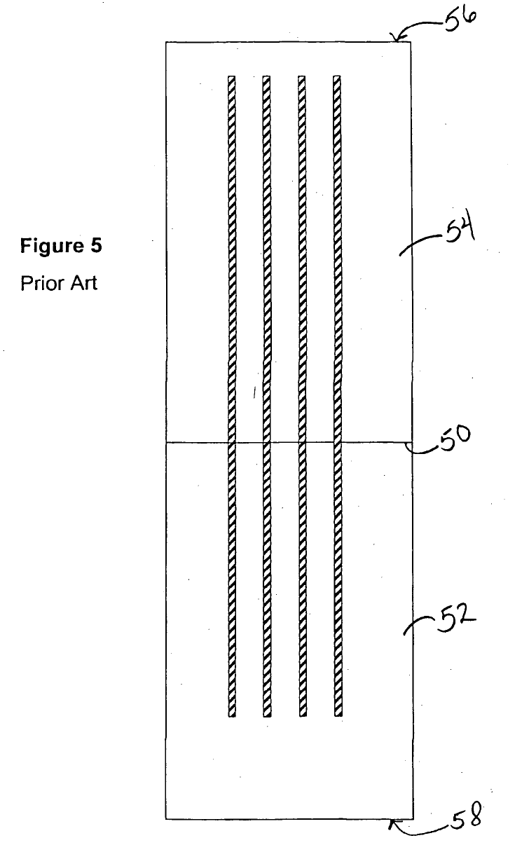

Table 1 provides skin care composition data for some current products (e.g., such as illustrated in FIG. 5). PAMPERS Baby Dry diapers, PAMPERS Custom Fit diapers and LUVS diapers are manufactured and sold by the Procter & Gamble Co. of Cincinnati, Ohio. Cutie diapers are sold by the Procter & Gamble Co. in Korea. Pequenin diapers are sold by Familia Sancella del Pacifico of Colombia. HUGGIES Ultratrim diapers and HUGGIES Supreme diapers are manufactured and sold by Kimberly-Clark Corporation of Neenah, Wis. As can be seen from Table 1, current designs employ a high front/back ratio, greater than 0.80, with greater than 1.10 more common, and even greater than 1.3 employed.

TABLE 1

| | Area % | Area % | Area | |
|---|---|---|---|---|
| | Covered | Covered | Front/back | Density |
| Designs | Front Half | Back Half | Ratio | (g/mm²) |
| PAMPERS Baby Dry | 12% | 9% | 1.33 | 1.41 × 10⁻⁵ |
| Diapers | | | | |
| LUVS Diapers | 10% | 9% | 1.11 | 1.14 × 10⁻⁵ |
| PAMPERS Custom | 11% | 8% | 1.38 | 1.18 × 10⁻⁵ |
| Fit Diapers | | | | |
| Pequenin Diapers | 7% | 6% | 1.17 | 1.07 × 10⁻⁵ |
| Cutie Diapers | 9% | 11% | 0.82 | 2.26 × 10⁻⁵ |
| HUGGIES Ultratrim | 9% | 8% | 1.13 | 2.19 × 10⁻⁵ |
| Diapers | | | | |
| HUGGIES Supreme | 9% | 8% | 1.13 | 3.27 × 10⁻⁵ |
| Diapers | | | | |

Table 2 shows data for the various exemplary designs set forth in Figures. As can be seen from Table 2, the designs set forth in this application have a substantially lower front/back ratio. Generally, a ratio of less than or equal to 0.70 is employed, with less than or equal to about 0.60 desirable, less than or equal to about 0.45 more desirable, less than or equal to about 0.35 employed, and less than or equal to about 0.15 even possible. As is illustrated in a comparison of Table 1 and Table 2, the designs of Table 2 have a higher percentage of covered area in the back. Generally, the current designs in Table 1 employ less than 12% covered back area, while the exemplary designs of Table 2 employ greater than 15% covered back area, with greater than or equal to about 20% covered back area desirable, greater than or equal to about 30% covered back area more desirable, and greater than or equal to about 40% covered back area even more common.

TABLE 2

| | Area % | Area % | Area |
|---|---|---|---|
| | Covered | Covered | Front/ |
| | Front | Back | Back |
| Designs | Half | Half | Ratio |
| Offset (FIG. 6) | 8% | 19% | 0.42 |
| Zoned Back (FIG. 7) | 7% | 46% | 0.15 |
| Shaped Zoned Back (FIG. 8) | 0% | 47% | 0.00 |
| Zoned Front/Back (FIG. 9) | 10% | 43% | 0.23 |
| Shaped Zoned Front/Back (FIG. 10) | 14% | 46% | 0.30 |
| Shaped Zoned Front/Back Matrix (FIG. 11) | 7% | 23% | 0.30 |

The absorbent article, e.g., diaper, disclosed herein has the advantage of improving skin care composition transfer to the wearer while minimizing interference with intake. In some embodiments, the composition area coverage is non-uniform, thereby further enhancing absorption and intake rate. Additionally, the current designs reduce migration into the absorbent core. By controlling the location of the skin care product, interference with the intake, distribution, absorption and retention of the insult is minimized or eliminated and a more effective transfer of the skin care composition is achieved (i.e., the composition is transferred to the affected area of the skin of the wearer), thereby enabling effective, efficient, prevention and/or treatment of red, irritated, and/or sore skin. Generally, the treatment is attained without requiring the manual application of messy, inconvenient lotions, ointments, or the like.

Additionally, the designs disclosed herein enable targeted skin care treatment focused on the targeted area, and various areas of skin can be treated differently. Different skin care compositions may be applied to different areas of the absorbent article to enhance the skin treatment/protection benefit with the type of skin care composition. Additionally, the density of skin care composition can also be varied in different areas of the bodyfacing surface. These can be optimised, based upon the area of the skin to be contacted, the type of insult (if any) received by that area, and/or the type of skin care issue generally affecting that area.

While the invention has been described in detail with respect to the specific aspects thereof, it will be appreciated that those skilled in the art, upon attaining an understanding of the foregoing, may readily conceive of alterations to, variations of, and equivalents to these aspects. Accordingly, the scope of the present invention should be assessed as that of the appended claims and any equivalents thereto.

1. An absorbent article having a front half extending from a front edge to a longitudinal centerline, a back half extending from a back edge to a longitudinal centerline, and a width defined by a narrowest part of the absorbent article, the absorbent article comprising:
an outer cover;
a liquid permeable bodyside liner that is connected in superposed relation to the outer cover;
an absorbent body that is located between the bodyside liner and the outer cover, on a side of the bodyside liner opposite a bodyfacing surface; and
a skin care composition on at least a portion of the bodyfacing surface;
wherein the skin care composition has a density of less than or equal to about 3.4×10⁻⁵ g/mm²; and
wherein the absorbent article has a front/back ratio of skin care composition of less than or equal to 0.70.

2. The absorbent article of claim 1, wherein the ratio is less than or equal to 0.60.

3. The absorbent article of claim 2, wherein the ratio is less than or equal to 0.45.

4. The absorbent article of claim 3, wherein the ratio is less than or equal to 0.35.

5. The absorbent article of claim 1, wherein the density is from 2.4×10⁻⁵ g/mm² to 3.1×10⁻⁵ g/mm².

6. The absorbent article of claim 5, wherein the density is from 2.8×10⁻⁵ g/mm² to 3.0×10⁻⁵ g/mm².

7. The absorbent article of claim 6, wherein the density is from 2.8×10⁻⁵ g/mm² to 2.9×10⁻⁵ g/mm².

8. The absorbent article of claim 1, wherein the density is less than or equal to 2.8×10⁻⁵ g/mm².

9. The absorbent article of claim 1, wherein a covered area of the back half covered with skin care composition is greater than 15%.

10. The absorbent article of claim 9, wherein the covered area is greater than or equal to 20%.

11. The absorbent article of claim 10, wherein the covered area is greater than or equal to 30%.

12. The absorbent article of claim 11, wherein the covered area is greater than or equal to 40%.

13. The absorbent article of claim 1, wherein the skin care composition comprises at least two different compositions.

14. The absorbent article of claim 13, wherein the compositions are located on different portions of the bodyfacing surface.

15. The absorbent article of claim 1, wherein the skin care composition has different densities in different areas of the bodyfacing surface.

16. An absorbent article having a front half extending from a front edge to a longitudinal centerline, a back half extending from a back edge to the longitudinal centerline, and a width defined by the narrowest part of the absorbent article, the absorbent article comprising:
an outer cover;
a liquid permeable bodyside liner that is connected in superposed relation to the outer cover;
an absorbent body that is located between the bodyside liner and the outer cover, on a side of the bodyside liner opposite a bodyfacing surface; and
a skin care composition on at least a portion of the bodyfacing surface;
wherein the skin care composition has a density of less than or equal to 3.4×10⁻⁵ g/mm²; and
wherein the skin care composition covers greater than or equal to 15% of an area of the back half.

17. The absorbent article of claim 16, wherein the article has a front/back ratio of skin care composition of less than or equal to 0.45.

18. The absorbent article of claim 17, wherein the article has a front/back ratio of skin care composition of less than or equal to 0.35.

19. The absorbent article of claim 16, wherein the density is from 2.4×10⁻⁵ g/mm² to 3.1×10⁻⁵ g/mm².

20. The absorbent article of claim 19, wherein the density is from 2.8×10⁻⁵ g/mm² to 3.0×10⁻⁵ g/mm².

21. The absorbent article of claim 20, wherein the density is from 2.8×10⁻⁵ g/mm² to 2.9×10⁻⁵ g/mm².

22. The absorbent article of claim 16, wherein the density is less than or equal to 2.8×10⁻⁵ g/mm².

23. The absorbent article of claim 16, wherein the skin care composition covers greater than or equal to 20% of the area of the back half.

24. The absorbent article of claim 23, wherein the skin care composition covers greater than or equal to 30% of the area of the back half.

25. The absorbent article of claim 24, wherein the skin care composition covers greater than or equal to 40% of the area of the back half.

26. The absorbent article of claim 16, wherein the skin care composition comprises at least two different compositions.

27. The absorbent article of claim 26, wherein the compositions are located on different portions of the bodyfacing surface.

28. The absorbent article of claim 16, wherein the skin care composition has different densities in different areas of the bodyfacing surface.
